(19) United States
(12) Patent Application Publication
(10) Pub. No.: US 2017/0007524 A1
(43) Pub. Date: Jan. 12, 2017

(54) PERSONALIZING SUBSTANCE FOR APPLICATION TO THE SKIN OR ADDITION TO TATTOO INK AND METHODS OF PREPARATION THEREOF

(71) Applicant: Chamber Works, LLC, Wilmington, DE (US)

(72) Inventors: Patrick Duffy, SR., Wilmington, DE (US); Patrick Duffy, JR., Wilmington, DE (US); Sherry Freebery, Wilmington, DE (US); Ellen Jorgensen, Brooklyn, NY (US)

(21) Appl. No.: 15/206,981

(22) Filed: Jul. 11, 2016

Related U.S. Application Data
(60) Provisional application No. 62/190,632, filed on Jul. 9, 2015.

(51) Int. Cl. A61K 8/60; A61Q 1/02; A61K 8/02

(52) U.S. Cl. CPC A61K 8/606; A61K 8/0241; A61Q 1/025; A61K 2800/43; A61K 2800/91; A61K 2800/413

(57) ABSTRACT

Compositions for delivering personalizing substances, such as a biological material, sand, soil, metal, water, sea water, holy water, synthetic or biological polymers, ceramics, animal or plant tissue, or another physiologically compatible component having personal significance to an individual are described herein. The compositions include the personalizing substance in combination with a pharmaceutically acceptable carrier for injection, such as tattoo ink. Also disclosed are methods for administering a personalizing substance to a recipient. In some embodiments, the personalizing substance is added to a tattoo ink and incorporated in a tattoo created on an individual's skin.

### CROSS REFERENCE TO RELATED APPLICATION

This application claims benefit of and priority to U.S. Provisional Patent Application No. 62/190,632 filed on July 9, 2015, incorporated by reference in its entirety.

### FIELD OF THE INVENTION

The present invention relates to personalizing materials to be added to the skin, such as additives for tattoo ink.

### BACKGROUND OF THE INVENTION

Humans have been applying tattoos to the skin for thousands of years. For example, the first recorded formula for mixing and applying tattoo ink dates back to the fifth century and is attributed to the Roman physician Aetius. Tattoo inks were derived from natural substances and comprised a suspension of pigmented particles in a liquid carrier. Applying tattoo ink with needles or similar instruments to the skin, where the ink remains permanently, produces tattoos. This technique introduces the pigment suspension through the skin by an alternating pressure-suction action caused by the elasticity of the skin in combination with the movement of the needle. Water and other carriers for the pigment introduced into the skin diffuse through the tissues and are absorbed. Once the skin has healed, most pigment particles remain in the interstitial space of the tissue. During the healing process, some particles of pigment are eliminated from the skin surface. After healing, the tattoo is made up of the remaining particles of pigment located in the dermis where they are engulfed by phagocytic skin cells or are retained in the extracellular matrix. See US Published application no. 2009/0311295 to Mathiowitz et al. Inks used for tattooing resist elimination due to their inertness and the relatively large size of the insoluble pigment particles. A tattoo produced in this manner will partially fade over time but will generally remain visible. Tattoos are used for a variety of reasons, primarily for ornamentation of the skin. See U.S. Pat. No. 6,013,122 to Klitzman & Koger.

Despite advances in methods of applying tattoo ink to the skin, such as the electric tattoo ink gun, tattoo inks in commercial use today are similar to those used centuries ago. Standard tattoo inks contain a pigment comprising metal salts dissolved in a carrier, usually ethanol or water, to disperse the pigment in the dermis. See U.S. Pat. No. 6,013,122. Thus a need exists for novel formulations of tattoo ink with improved properties.

### SUMMARY OF THE INVENTION

Compositions for delivering materials, such as a biological material, sand, soil, metal, water, sea water, holy water, synthetic or biological polymers, ceramics, animal or plant tissue, or another physiologically compatible component having personal significance to an individual (herein after, personalizing substance) are described herein. In certain embodiments, the personalizing substance is in the form of nanoparticles. Alternatively, it could be microparticulate in size, having a size ranging from 1 micron to 5 microns. In a preferred embodiment, the personalizing substance is not encapsulated in a polymeric nano- or microparticle, eliminating some additional costly processing steps of microencapsulation (other than micronization for example to provide the personalizing substances in an appropriate size range for injection) prior to administering the personalizing substance to a recipient. In certain embodiments, the composition further comprises a carrier suitable for injection into the skin. For example, the compositions include a personalizing substance in combination with tattoo ink, creating a personalized tattoo ink. A personalized ink tattoo creates a physical connection with a person, object, place, or event, because the personalized tattoo incorporates into the tattoo ink and, therefore, the image displayed in the skin, a personalizing substance.

A preferred personalizing substance is DNA, which can further include a personal identification characteristic selected from the group consisting of short tandem repeats (STRs), single nucleotide polymorphisms (SNPs), epigenetic markers, and methylated DNA patterns. The DNA can be obtained from one or more humans, non-human animals, or plants of significance to the individual, or any combination thereof. The DNA may be obtained by any standard method, such as a non-invasive cheek-swab. In a preferred embodiment, the personalizing substance does not contain cremated ash. In some embodiments, a personalizing substance may be combined with cremated ash and administered to an individual's skin. In certain embodiments of the compositions described herein, the personalizing substance is DNA, and the composition includes up to 0.01% (w/w) DNA. In certain embodiments, the personalizing substance is not DNA, and the carrier includes up to 10% (w/w) of the personalizing substance.

The personalizing substance may be delivered to a person's skin to create a personalized ink tattoo. For example, the personalizing substance may be additives to a tattoo ink. Alternatively, the personalizing substance can be delivered to the individual's skin without adding a tattoo ink, for example, by administering the personalizing substance to a site where a tattoo is already present.

The personalizing substance is delivered to the individual in a suitable carrier, optionally in combination with a tattoo ink. The personalizing substance may be mixed with the tattoo ink and the mixture can be applied using a standard tattoo needle and procedure. In other embodiments, the injection is made at the site of an existing tattoo.

In some embodiments, the method further comprises mixing the personalizing substance with a tattoo ink prior to injection. Alternatively, the personalizing substance is administered with a suitable carrier (i.e., which is not tattoo ink) to one or more desired sites in an individual's skin. Typically, a marker is added to the site, or the site contains a marker, to indicate the location of the injected material. In certain embodiments, the injection is made at the site of an existing tattoo as an additive composition.

### BRIEF DESCRIPTION OF THE DRAWINGS

FIG. 1 is a flow chart showing the steps of an exemplary end-to-end process for delivering to a customer a tattoo that incorporates DNA of personal significance. The steps performed by the customer and then by the lab generate DNA, alone (dry powder), or in liquid form (e.g. with a carrier solution), which is added as an additive to a tattoo ink.

FIG. 2 is a flow chart showing the steps of an exemplary end-to-end process for delivering to a customer a tattoo that incorporates one or more compounds of personal significance. The steps performed by the customer and then by the lab generate compound(s) of personal significance, alone (dry powder), or in liquid form (e.g. with a carrier solution), which is added as an additive to a tattoo ink.

### DETAILED DESCRIPTION OF THE INVENTION

### I. Definitions

As used herein, the term “personalizing substance” refers to a material of significance to an individual. The personalizing substance may be a natural or synthetic material, and can be micronized.

The terms “additive” and “additive composition” are used interchangeably herein to refer to a composition that is added to an existing tattoo or to a tattoo ink, with or without a carrier, prior to or during, tattooing.

As used herein, the terms “carrier” or “additive carrier” mean a composition for dissolving or storing a personalizing substance for administration to a recipient. The carrier is typically suitable for injection into the human skin.

As used herein, the term “biological material” means any biological substance, including, but not limited to biological micromolecules, such as a nucleotides, amino acids, cofactors, or hormones, biological macromolecules, such as nucleic acids, polypeptides, proteins (for example enzymes, receptors, secretory proteins, structural and signaling proteins, hormones, ligands, etc.), polysaccharides, and/or any combination thereof.

As used herein, the term “physiologically compatible component” means any component in a composition that is compatible with the physiology of the recipient, typically a human.

As used herein, the term “recipient” refers to the recipient of the personalizing substance. The recipient may be any subject, human, animal or plant, capable of receiving the personalizing substance.

As used herein, “nanoparticle” refers to a particle or a structure in the nanometer (nm) range, typically from about 1 to about 1000 nm in diameter.

“Micronization” is a term used to describe size reduction where the resulting particle size is less than 10 microns.

As used herein, a “microparticle” is a particle of a relatively small size, but not necessarily in the micron size range; the term is used in reference to particles of sizes that can be, for example 1 to about 1000 microns. The term “microparticle” encompasses microspheres, microcapsules and microparticles, unless specified otherwise. A microparticle may be of composite construction and is not necessarily a pure substance; it may be spherical or any other shape.

### II. Compositions

Compositions for placing a personalizing substance in the skin of an individual, to remain at the site of placement, are described herein. The compositions may be used to personalize tattoos and/or integrate substances of special significance to an individual into his/her skin. The compositions include a personalizing substance in combination with a carrier for injection into the skin. In one embodiment, the carrier is tattoo ink. However, the carrier can be any pharmaceutically acceptable carrier suitable for injection.

Obtaining a personalized ink tattoo creates a physical connection with a person, object, place or event, because the personalized tattoo incorporates into the tattoo ink and, therefore, the image displayed in the skin, a personalizing substance. The personalizing substance should be non-immunogenic in the recipient when applied to the skin.

In a preferred embodiment, the personalizing substance is not encapsulated in a polymeric nano- or microparticle or matrix, eliminating costly processing steps of microencapsulation (other than micronization for example to provide the personalizing substances in an appropriate size range for injection) prior to administering the personalizing substance to a recipient.

1. Personalizing Substance

Suitable personalizing substances include, but are not limited to, biological materials such as, for example, animal or plant tissue, sand, soil, metal, sea water, holy water, synthetic or natural polymers, ceramics, and other physiologically compatible components.

The personalizing substance is preferably not a component in conventional tattoo inks. For example, in a preferred embodiment, the personalizing substance is not a chromophore found in conventional tattoo inks (disclosed below). In those embodiments where some amount of the personalizing substance is present in a conventional tattoo ink, the compositions disclosed herein can be distinguished from such conventional tattoo inks as they contain personalizing substance at levels above those present in a conventional tattoo ink, i.e., tattoo inkper se is not a personalizing substance as defined herein. However, tattoo ink can be used as a carrier for personalizing substances.

In a more preferred embodiment, the personalizing substance is not a metal or metal oxide as used in tattoo ink.

In the case of liquid personalizing substances such as sea water and holy water, lyophilization of microparticles comprising the personalizing substance would remove any liquid contained in the microparticle. However, any salts or other non-volatile compounds contained in the liquid would remain.

In some embodiments, the compositions include DNA without any additional personalizing substances. In other embodiments, the compositions contain a personalizing substance comprising DNA and one or more additional personalizing substances comprising other compounds. For example, the additional personalizing substances may be one or more samples from sand, soil, metal, ceramics, and/or plant products.

The identity of personalizing substances that do not contain DNA, such as sand, soil, metal, water, sea water, holy water, synthetic or natural polymers, ceramics, and compounds derived from plants, may be confirmed by a suitable method, such as mass spectrometry, for example, isotope-ratio mass spectrometry (IRMS) or liquid chromatography mass spectrometry (LC-MS).

### (a) Non Biological Personalizing Substances

Sand consists predominately of silica (SiO₂) and other organic and inorganic minerals, such as calcium silicate (Ca₂SiO₄), calcium nitride (Ca₃N₂), silicon nitride (Si₃N₄), aluminum nitride (A1N₃), alumina (Al₂O₃), borazone “boron nitride” (BN), magnesium oxide (MgO), silicon oxysulfide (SiOS), lithium silicate (Li₂SiO₄), as well as other metal oxides/nitrides, as shown in Table 1.

TABLE 1
Exemplary Personalizing Substances

| Source for | |
|---|---|
| Personalizing | |
| Substance | Personalizing Substance |
| White Beach Sand | Quartz (SiO₂) particles of different diameter ranges |
| | and limestone from coral or shells. |
| Dark Sand | Quartz (SiO₂) particles of different diameter ranges |
| | and magnetite. |
| Green Sand | Quartz (SiO₂) particles of different diameter ranges |
| | and chlorite |
| Rock | Quartz (SiO₂) particles of different diameter ranges |
| | and other trace elements that vary with geographical |
| | location. |

For example, the personalizing substance may contain silicon dioxide particles extracted from a soil or rock sample. Suitable extraction techniques are known. Following extraction, the particles may be ground by conventional means to reduce their size to less than 1 micron, optionally the particles are then screened to obtain a population of particles having a the desired size range for injection, or micronized to produce nanoparticles of suitable size, typically from about 1 to about 1000 nm in diameter.

In some embodiments, the personalizing substance comprises particles of a metal or ceramic object having meaning to a person receiving the substance. For example, such metal or ceramic objects can be ground, screened and extracted to remove unwanted components, then mixed with tattoo ink for inclusion in a tattoo.

In some embodiments, the personalizing substance includes extracts of wooden items that have personal meaning to the individual. For example, in some embodiments cellulose is extracted from the wood item and mixed with tattoo ink delivery to the individual.

### (b) Types of DNA Molecules in Personalizing Substances

The personalizing substances are intended to remain inert and unreactive following delivery to the skin. Accordingly, the personalizing substance preferably does not comprise a vector. As used herein the term “vector” refers to a DNA molecule used in biotechnology for storage, propagation, delivery or integration of recombinant DNA. Examples of vectors include plasmid backbones, viral vectors, bacmids, cosmids, and artificial chromosomes. Generally, the vector itself is a DNA sequence that consists of an insert (transgene, or recombinant DNA) and a larger sequence that serves as the “backbone” of the vector. The purpose of a vector is to transfer the insert to another cell, where it may be isolated, multiplied, or expressed. In some embodiments, the personalizing substance does not comprise DNA that is used to transfer a DNA sequence into a cell. In some embodiments, the personalizing substance does not comprise DNA used for the purpose of multiplying or expressing the genetic information contained within it.

In certain embodiments, the personalizing substance to be delivered to the individual contains DNA from a human, a non-human animal (e.g. a pet), or a plant.

In a particular embodiment, the DNA is from a human. No two people have the exact same sequence of DNA in their cells. The differences in the DNA in individual humans gives rise to the unique DNA profiles that can be used to distinguish individuals. In addition, the unique DNA profile of each individual provides a means for verifying that the personalizing substance is from a particular individual. Accordingly, incorporation of DNA into a carrier or into a tattoo ink provides a unique characteristic to the tattoo ink or carrier that may be verified, for example, through DNA sequencing or analysis of genetic markers.

The DNA may be coding or non-coding genomic DNA, coding or non-coding mitochondrial DNA or complementary DNA (cDNA). cDNA is synthesized from RNA using reverse transcriptase. The genomic DNA, mitochondrial DNA, and RNA for synthesis of cDNA may be isolated from any organism, including but not limited to humans, animals, and plants. In some embodiments, the DNA is isolated from a single organism, for example, a human. In other embodiments, the DNA is isolated from two or more organisms, for example, two or more humans. Methods of isolating genomic DNA, mitochondrial DNA and RNA, and methods of cDNA synthesis are well known in the art and are described, for example, in Sambrook, et al., Molecular Cloning. (4th ed.). Cold Spring Harbor, N.Y.: Cold Spring Harbor Laboratory.

### i. DNA Isolation and Amplification

In some embodiments, the DNA contained in the personalizing substance is isolated directly from an organism, such as genomic DNA or mitochondrial DNA. In other embodiments, the DNA contained in the personalizing substance is amplified from a sample collected from the organism, for example by polymerase chain reaction (PCR). Multiple DNA segments for tetranucleotide PCR amplification typically may be amplified in a single tube. Such multiple amplification of several DNA regions is known in the art as multiplex PCR. The multiple PCR products are separated as known in the art, for example, by electrophoresis, and an instrument reads the electrophoresis gel or image to automatically analyze the sizes of the PCR products. In some embodiments, the DNA contained in the personalizing substance is cDNA reverse transcribed from RNA isolated from the organism, as mentioned above.

The DNA may be sequenced so that verification steps described below may be performed. (Sambrook, et al., Molecular Cloning. (4th ed.). Cold Spring Harbor, N.Y.: Cold Spring Harbor Laboratory).

Preparation of DNA samples for use as a personalizing substance may proceed as follows, although other methods of preparing analogous DNA samples are known to the skilled artisan. One preferred method includes the following general steps: A sample for preparation of the DNA contained in the personalizing substance is collected from a sample of cheek swab, skin, hair, saliva, or blood or other tissue from an organism as is known in the art. A cheek swab sample is preferred. Protocols for collecting and handling the sample are known in the art.

For example, a DNA isolation kit suitable for isolating genomic DNA from buccal cells, may be used to isolate DNA from the cheek swab. These kits are commercially available and usually generate 0.5-2 micrograms of total DNA. Desirable genomic regions containing polymorphic genetic markers (such as STRs and SNPs) of the isolated DNA are then amplified via PCR to generate micrograms, typically from 1 to 10 micrograms, of DNA to be used as a personalizing substance. The amplified DNA may be sequenced so that verification steps described below may be performed. This amplified DNA is the personalizing substance that is administered to a recipient.

In some embodiments, genomic DNA, mitochondrial DNA, and/or RNA is isolated from the sample using methods known in the art, such as those described in Sambrook et al. (cited above). The concentration and integrity of the extracted DNA or RNA may be determined, for example, to inform the decision to proceed with PCR or reverse transcription or to obtain another sample. The DNA contained in the personalizing substance may also be generated by PCR. For example, DNA including STRs may be amplified by PCR using primers that amplify three to five tetranucleotide repeat segments of the genomic DNA sample, optionally incorporating a detectable label, such as a radioactive or fluorescent label, as is known in the art. PCR primers for amplifying the DNA may be obtained from a commercial source or may be synthesized using methods known in the art. Software for design of PCR primers is well known in the art.

Examples of preferred STRs that may be amplified by PCR are set forth in Table 2 below. The skilled artisan will appreciate that additional suitable tetranucleotide and pentanucleotide repeats may also be amplified. One of the preferred qualities of suitable tetranucleotide DNA repeats is high heterozygosity (variability between individuals) in the subject population. Another preferred quality of suitable tetranucleotide DNA repeats is that they do not encode a biologically active product, for example, a protein, tRNA, rRNA, miRNA, or siRNA. A further preferred quality of suitable tetranucleotide DNA repeats is that they do not induce an immune response and produce no therapeutic action in the recipient.

TABLE 2
Preferred Repeats in DNA for amplification

| Human | Allele Distribution | Number of |
|---|---|---|
| Marker | (bp) | Repeats |
| 3S1358 | 98 to 146 | 8 to 20 |
| 5S818 | 133 to 169 | 7 to 16 |
| 7S820 | 215 to 247 | 6 to 14 |
| 8S1179 | 163 to 213 | 7 to 19 |
| 13S317 | 161 to 205 | 5 to 16 |
| 16S539 | 133 to 173 | 5 to 15 |
| 21S11 | 201 to 257 | 24 to 38 |
| 8S1106 | 109 to 133 | 7 to 13 |
| 1S518 | 182 to 198 | 13 to 17 |
| 6S1017 | 354 to 374 | 10 to 15 |
| 17S1304 | 197 to 213 | 10 to 14 |
| 4S2408 | 336 to 360 | 13 to 19 |
| 5S1467 | 173 to 189 | 8 to 12 |
| 19S245 | 225 to 249 | 16 to 22 |

The resulting PCR products are typically analyzed, for example, by electrophoresis, for the successful generation of tetranucleotide repeats and to confirm that the sample shows relatively unique representation of a DNA sample from an individual.

### ii. Verification of amplified DNA

DNA can be analyzed to confirm that the DNA contained in the personalizing substance was obtained or generated from the desired source organism. For example, for DNA including STRs, the pattern of PCR products in the DNA contained in the personalizing substance may be compared to a control sample obtained from the source organism. The DNA contained in the personalizing substance may also be analyzed by DNA sequencing, for example cDNA sequencing or whole genome sequencing, to confirm that the DNA contained in the personalizing substance is from the desired source organism.

The sequencing of the DNA may be performed using other methods known in the art. These include, but are not limited to basic sequencing methods, such as Sanger's method, Maxam-Gilbert sequencing and chain termination methods (Franca et al., Quarterly Review of Biophysics, 35(2):169-200, 2002), advanced methods and de novo sequencing, such as shotgun sequencing and bridge PCR (Braslavky et al., Proc. Natl. Acad. Sci, 100(7):3960-3964, 2003), or next-generation methods. Next-generation sequencing applies to genome sequencing, genome resequencing, transcriptome profiling (RNA-Seq), DNA-protein interactions (ChIP-sequencing), and epigenome characterization (de Magalhäes et al., Ageing Res Rev. 9(3)315-323, 2010; Liu et al., Journal of Biomedicine and Biotechnology, 2012:1-11, article ID 251364, 2012; and Hall, The Journal of Experimental Biology, 209:1518-1525, 2007). Resequencing is necessary, because the genome of a single individual of a species will not indicate all of the genome variations among other individuals of the same species. Next Generation sequencing encompasses a number of methods, including, but not limited to single-molecule real-time sequencing, massively parallel signature sequencing, (MPSS), Polony sequencing, 454 pyrosequencing, ion torrent semiconductor sequencing, DNA nanoball sequencing, heliscope single molecule sequencing, sequencing by ligation (SOLiD sequencing) and single molecule real time sequencing (SMRT). These methods are detailed and compared in Liu et al., Journal of Biomedicine and Biotechnology, 2012:1-11, article ID 251364, 2012, and Hall, The Journal of Experimental Biology, 209:1518-1525, 2007.

In some embodiments, the DNA contained in the personalizing substance is analyzed before the personalizing substance is combined with a carrier or in tattoo ink. In other embodiments, the DNA contained in the personalizing substance is analyzed after the personalizing substance is combined with a carrier, optionally in combination with tattoo ink. In some embodiments, the carrier is tattoo ink.

The DNA may be purified to obtain pharmaceutical/biologics grade DNA suitably free of contaminants.

(c) Shapes and Sizes

Any of the aforementioned personalizing substances may be micronized where appropriate, to produce nanoparticles of suitable size using methods known in the art (Joshi, J. Pharmaceutical Sci. and Technol., 3(7):651-681 (2011). The diameter of the nanoparticle may be, for example, about 1000, 900, 800, 700, 600, 500, 400, 300, 200, 100, 90, 80, 70, 60, 50, 40, 30 or 20 nanometers (nm). In certain embodiments, the diameter of the nanoparticle is less than about 1000, 900, 800, 700, 600, 500, 400, 300, 200, 100, 90, 80, 70, 60, 50, 40, or 30 nanometers (nm). Any of these values may be used to define a range for the diameter of the nanoparticle. For example, the diameter of the nanoparticle may be from about 20 nm to about 1000 nm or from about 20 nm to about 100 nm.

The personalizing substances may have the size of microparticles and can have any shape. Typically the microparticles are spherical. Other suitable shapes include, but are not limited to, flakes, triangles, ovals, rods, polygons, needles, tubes, cubes and cuboid structures. In certain embodiments, the microparticles have a diameter of less than 10, 9, 8, 7, 6, 5, 4, 3, 2, 1, 0.9, 0.8, 0.7, 0.6, 0.5, 0.4, 0.3, 0.2 or 0.1 micron(s). Any of these values may be used to define a range for the diameter of the microparticle. For example the diameter of the microparticle may be from about 0.1 to about 10 microns, from about 0.1 to about 1 micron, or from about 0.1 to about 2 microns. Typically, the microparticle diameter is less than 5 microns. Preferably for compositions that are used as additives in a tattoo ink, the microparticle diameter ranges from about 1 to about 10 microns, more preferably from about 1 to 2 microns.

Microparticles with a diameter of 10 microns and less may be introduced into the skin via a tattoo gun or any similar device. In other embodiments, larger microparticles or particles may be used. For example the microparticles may have a diameter of ranging from 10 microns to 1000 microns. In these embodiments, the microparticles may be delivered to the skin via an intradermal injection.

2. Carriers

The personalizing substance can be included in a suitable pharmaceutically acceptable carrier for injection. In some embodiments, the cells are simply suspended in a physiological buffer. In a preferred embodiment, the carrier is tattoo ink.

Tattoo ink consists of pigments combined with a carrier, and used for tattooing. The oldest pigments came from using ground up minerals and carbon black. Today's pigments include the original minerals, modern industrial organic pigments, and some vegetable-based pigments. Some pigments are provided as follows: black (Fe₃O₄, FeO, Caron, logwood; Brown (Ochre- which is ferric oxides mixed with clay; red (cinnabar, Cadmium, red, Fe₂O₃; Napthol-AS pigment); Orange(disazordiarylide and/or disazopyrazolone, cadmium seleno-sulfide); yellow (cadmium yellow, curcuma yellow, chrome yellow, etc); green (Cr₂O3, malachite, Ferrocyanides and ferricyanides, lead chromate, monoazo pigment, etc); white (lead white, titanium oxide, barium sulfate, zinc oxide). Other pigments and chromophores that can be included in tattoo ink, are disclosed for example in U.S. Publication No. 2009/0311295.

In some embodiments the composition includes a suitable biocompatible carrier which is not tattoo ink, for delivery to a human via injection. Suitable carriers include any alcohol, including but not limited to ethyl alcohol, isopropyl alcohol, or water. Suitable carriers also include any combination of alcohol and water. Typically the amount of alcohol in the carrier ranges from about 5% to about 30% (w/w), and the amount of water in the carrier ranges from about 40% to about 70% (w/w).

In preferred embodiments, the carrier is a solution of 60% water, 30% glycerin (glycerol), and 10% ethanol. Other carrier solutions including 55% water, 30% glycerin and15% ethanol; 50% water, 30% glycerin, and 20% ethanol; 45% water, 30% glycerin, and 25% ethanol; or 40% water, 30% glycerin and 30% ethanol, are also contemplated.

3. Optional Components

### Personal Identification Characteristics

Optionally, the compositions include one or more personal identification characteristics. The one or more personal identification characteristics contain unique information which can be used to verify that the personalizing substance was obtained from a particular source, e.g., human, non-human animal, or plant. Exemplary personal identification characteristics for DNA include, but are not limited to, microsatellite markers such as short tandem repeats (STRs) and Simple Sequence Repeat (SSR) markers, single nucleotide polymorphisms (SNPs), and epigenetic markers, such as methylated DNA patterns. Any DNA sequence that is unique to the source organism may be used as a personal identification characteristic. For example the DNA sequence unique to the source organism may be identified by sequencing the entire sequence of the DNA isolated from the source organism, or a portion thereof, using sequencing methods known in the art such as Sanger sequencing or next generation sequencing, e.g. Illumina sequencing. DNA sequencing methods are well known in the art and are described, for example, in Sambrook, et al., Molecular Cloning. (4th ed.). Cold Spring Harbor, N.Y.: Cold Spring Harbor Laboratory.

### a. Polymorphic Genetic Markers

DNA generally includes one or more polymorphic genetic markers. Polymorphic genetic markers are highly variable regions of the genome which have contributed to the development of a variety of applications such as forensic DNA analysis and paternity testing that are used to unambiguously identify individuals.

The identification of many polymorphic genetic markers has occurred over the last thirty years. For example, polymorphic genetic markers known as variable number of tandem repeats (VNTRs) are abundant and highly polymorphic regions of DNA containing nearly identical sequences, 14 to 80 bases in length, repeated in tandem. See Jeffreys et al., 1985, Nature 314: 67-73; Wyman et al., 1980, PNAS 77: 6754-6758; and Nakamura et al., 1987, Science 235: 1616-1622. The variation in these markers between individuals makes them useful for identifying particular individuals. VNTRs may be detected from small amounts of DNA using polymerase chain reaction (PCR). See Kasai et al., 1990, Journal of Forensic Sciences 35(5): 1196-1200. Size differences in the amplified PCR products are detected on agarose or polyacrylamide gels. However, the finite number of VNTRs limits the widespread applicability of this method, which in turn led to the identification of short tandem repeats (STR).

### b. Short Tandem Repeats (STR)

STRs can be amplified by a polymerase chain reaction, and are highly abundant and polymorphic (variable from individual to individual). STRs can contain tandem repeat sequences that differ by two (dinucleotide), three (trinucleotide), four (tetranucleotide) or five (pentanucleotide) base pairs. It is estimated that there are approximately 50,000 to 100,000 dinucleotide repeats in the human genome. Trinucleotide and tetranucleotide repeats are less common; the human genome is estimated to contain 10,000 of each type of repeat. See Tautz et al, Nuc. Acids Res. 17: 6464-6471 (1989); and Hamada et al., PNAS 79:6465-6469 (1982). The use of tetranucleotide and pentanucleotide STRs allows better discrimination of differences between individual subjects relative to the shorter sequences. Weber et al., Am J Hum Genet, 44: 88-396 (1989).

The personalizing substance may contain a human DNA sequence selected from the group consisting of a dinucleotide STR, a trinucleotide STR, a tetranucleotide STR and a pentanucleotide STR.

Because the size of PCR products from human tetranucleotide repeat regions typically varies between individuals, tetranucleotide repeats are a preferred personal identification molecule for use as a personalizing substance. For example, PCR products of two different sizes are observed based on the inheritance for each individual of one copy of the polymorphic marker from each parent. Each inherited copy contains a variable number of tetranucleotide repeats. Thus, two unrelated individuals likely will produce different sized PCR products from the same tetranucleotide polymorphic marker. As a greater number of different tetranucleotide repeat regions are compared between individuals, the probability of those individuals sharing the identical pattern of PCR products decreases.

### c. Single Nucleotide Polymorphisms (SNPs)

Single nucleotide polymorphism is a DNA sequence variation occurring commonly within a population (e.g. 1%) in which a single nucleotide—A, T, C or G—in the genome (or other shared sequence) differs between members of a biological species or paired chromosomes. For example, two sequenced DNA fragments from different individuals, AAGCCTA to AAGCTTA, contain a difference in a single nucleotide.

SNPs may fall within coding sequences of genes, non-coding regions of genes, or in the intergenic regions (regions between genes). SNPs within a coding sequence do not necessarily change the amino acid sequence of the protein that is produced, due to degeneracy of the genetic code.

SNPs in the coding region are of two types, synonymous and nonsynonymous SNPs. Synonymous SNPs do not affect the protein sequence while nonsynonymous SNPs change the amino acid sequence of protein. The nonsynonymous SNPs are of two types: missense and nonsense.

SNPs that are not in protein-coding regions may still affect gene splicing, transcription factor binding, messenger RNA degradation, or the sequence of non-coding RNA. Gene expression affected by this type of SNP is referred to as an eSNP (expression SNP) and may be upstream or downstream from the gene.

SNPs without an observable impact on the phenotype (so called silent mutations) are still useful as genetic markers in genome-wide association studies, because of their quantity and the stable inheritance over generations.

III. Methods of making the Compositions

Methods for micronizing the personalizing substance for production of nanoparticles, if needed, include, for example, sonication and/or production of shear forces, and rotor stator mixing or milling with a concentric shaft, at a speed between, for example, 5,000 RPM and 25,000 RPM. Other methods are reviewed in Joshi, J. Pharm. Sci. Technol., 3(7):651-681 (2011).

In some embodiments where DNA is the personalizing substance, the DNA may be prepared by precipitation using standard techniques, such as ethanol or isopropanol precipitation, or salt precipitation. In some embodiments, the DNA is micronized by precipitation with calcium phosphate, and the precipitate is not dissolved but instead incorporated directly as nanoparticles into the microparticle. In a preferred embodiment, the micronized personalizing substance is not encapsulated in a polymeric nano- or microparticle, eliminating some additional costly processing steps of microencapsulation (other than micronization for example to provide the personalizing substances in an appropriate size range for injection) prior to administering the personalizing substance to a recipient. For example, the methods disclosed herein eliminations intermediary steps of microencapsulation such as solvent evaporation microencapsulation, hot melt microencapsulation, phase separation microencapsulation, spontaneous emulsification, melt-solvent evaporation, solvent evaporation microencapsulation, solvent removal microencapsulation, coacervation, multi-walled microencapsulation, all known methods employed to encapsulate/disperse a substance of choice in a polymeric nano-/microparticle.

### IV. Methods of Use

The personalizing substances described herein are preferably used in combination with a carrier, or as additives to substances, such as additives to tattoo ink, for delivery to the skin, typically via injection. The use of the composition may be chosen by the end user. For example, the compositions may be used by the end user to preserve a substance of personal significance for a long period of time. The end user may store the composition, or chose to present the composition as a gift to another individual. Alternatively, the end user may choose to use the composition as an additive to other substance.

In some embodiments, the compositions are administered via injection at a desired skin site of an individual. Typically, the site contains a marking or a marking is added to the site to indicate the presence of the administered personalizing substance in that location.

In a preferred embodiment, the personalizing substances may be used as an additive to tattoo inks. The additives may be used to create a type of tattoo having a physical connection with a person or place or event.

In certain embodiments, the personalizing substance is formulated in a dry powder form suitable for mixing with a carrier by the tattooist. The personalizing substance may be mixed with a carrier and supplied as a pre-dispersed solution. The tattoo ink used in combination with the personalizing substance may be of any desired color known in the art. Tattoo ink comprising a personalizing substance may be prepared by mixing microparticles that contain a personalizing substance with a tattoo ink that is suspended in a liquid such as water, glycerin or witch hazel. The personalizing substance nano- or microparticles and tattoo ink may be mixed by shaking, stirring, vortexing, or light sonicating of the microparticles with the tattoo ink. The concentration of the nano-/microparticles in the mixture of microparticles and tattoo ink is 0.001%, 0.005%, 0.01%, 0.05%, 0.1%, 0.5%, 1%, 2%, 3%, 4%, 5%, 6%, 7%, 8%, 9% or 10% w/w.

Once the personalizing substance particles are suspended within the tattoo ink, the tattoo ink may be administered to an individual by pouring a small amount (for example, less than 10 grams) of the microsphere-decorative tattoo ink slurry into a cup or other receptacle that is of sufficient size for one to dip a tattoo instrument that contains a tattoo needle or set of needles into the cup or receptacle. A tattoo may be created by dipping the tattoo needle into the cup or receptacle that contains the microparticle- tattoo ink mixture, and then piercing the skin with the needle to inject the microparticle-tattoo ink mixture into the skin.

### V. Kits

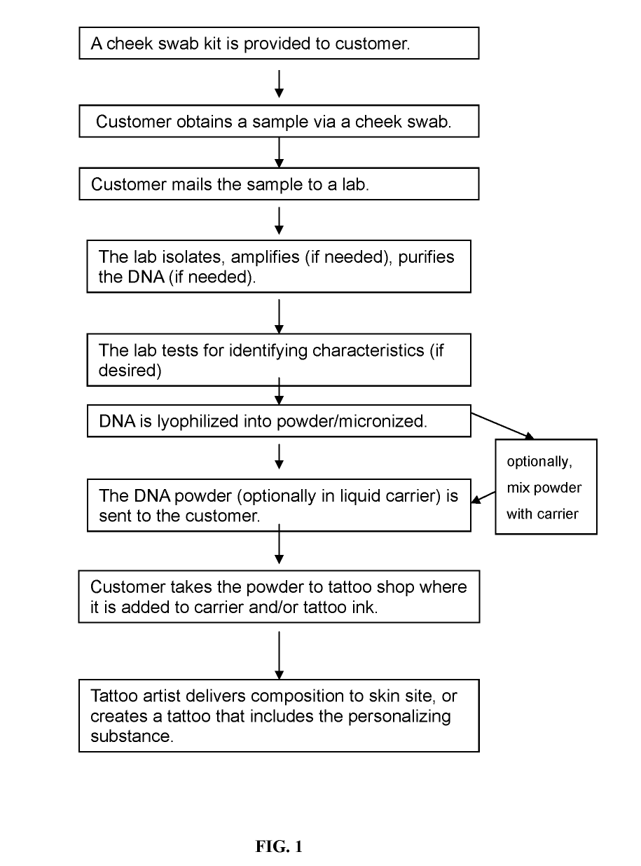

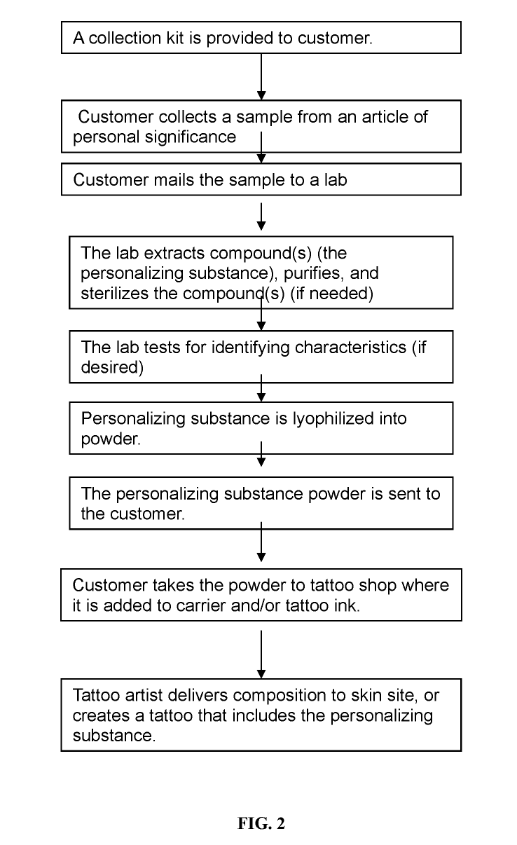

Kits for obtaining a personalizing substance from an end user and kits for delivering the personalizing substance to the end user are provided. A flow chart depicting the end-to-end process of obtaining the personalizing substance, and then isolating, preparing and delivering the personalizing substance to the end user is presented in FIG. 2. A similar flow chart with DNA used as an exemplary personalizing substance is presented in FIG. 1.

In the process illustrated in FIG. 1, a cheek swab kit is provided to a customer. The customer uses the cheek swab to obtain a sample from the human or non-human animal of interest to the customer. Then the customer mails or otherwise delivers the sample to a lab. The lab isolates, amplifies (if needed), and purifies (if needed) the DNA. The DNA or other personalizing substance is lyophilized into a powder. Optionally, the powder is added to a carrier suitable for injection (not shown). Then the powder or solution is delivered to the customer. The customer then delivers the solution or powder to a tattoo shop, which either injects it in a desired site in the customer or adds it to tattoo ink and creates a tattoo on the customer.

In the process illustrated in FIG. 2, a collection kit is provided to a customer. The customer places in the collection vessel (e.g. a vial) a sample from a source of interest to the customer. Then the customer mails or otherwise delivers the sample to a lab. The lab extracts, optionally purifies and/or sterilizes one or more compounds from the personalizing substance, and then reduces the substance (if needed), to a desired shape and size as disclosed herein. Then the personalizing substance may be lyophilized/micronized into a powder. Optionally, the powder is added to a carrier suitable for injection (not shown). Then the powder or solution is delivered to the customer. The customer then delivers the solution or powder to a tattoo shop, which either injects it in a desired site in the customer or adds it to tattoo ink and creates a tattoo on the customer.

The kits provide the equipment for obtaining a sample of the personalizing substance. The equipment may be tailored to the nature of the personalizing substance that will be provided. For example, if the personalizing substance is DNA, then the kit may include a foam or cotton-tipped cheek swab, a protective container for the swab, and instructions for use. If the personalizing substance is sand, then the kit may include a waterproof container and instructions for use.

In other embodiments, the kits provide the final product for use by the end user. In these embodiments, the kits may include a personalized substance, a carrier, and instructions for use. The kits may be customized to the preference of the end user. For example, the kits contain the personalizing substance in a powder form and a carrier. In other embodiments, the kits may contain the personalizing substance pre-mixed with the carrier or the personalizing substance in unmixed form for delivery to a tattoo shop for injection.

We claim:

1. A composition comprising a personalizing substance wherein the personalizing substance is not encapsulated in a polymeric nano- or microparticle, and a carrier suitable for injection into the skin.

2. The composition of claim 1, wherein the carrier is tattoo ink.

3. The composition of claim 1, wherein the personalizing substance is in the form of nanoparticles.

4. The composition of claim 1, wherein the personalizing substance is selected from the group consisting of isolated DNA, sand, soil, metal, ceramics, and plant tissue.

5. The composition of claim 4, wherein the personalizing substance is isolated DNA and wherein the DNA further comprises a personal identification characteristic selected from the group consisting of short tandem repeats (STRs), single nucleotide polymorphisms (SNPs), epigenetic markers, and methylated DNA patterns.

6. The composition of claim 4, wherein the personalizing substance is isolated DNA.

7. The composition of claim 1, wherein the carrier comprises alcohol and water.

8. The composition of claim 7, wherein the carrier comprises alcohol in a range from 5% to about 30% (w/w), and the amount of water in the carrier ranges from about 40% to about 70% (w/w).

9. The composition of claim 1, wherein the carrier is selected from the group consisting of a combination of 60% water, 30% glycerin, and 10% ethanol; a combination of 55% water, 30% glycerin and 15% ethanol; a combination of 50% water, 30% glycerin, and 20% ethanol; a combination of 45% water, 30% glycerin, and 25% ethanol; and a combination of 40% water, 30% glycerin and 30% ethanol.

10. The composition of claim 1, wherein the personalizing substance is not DNA, and wherein the carrier comprises up to 10% (w/w) of the personalizing substance.

11. The composition of claim 1 comprising the personalizing substance in a concentration range between 0.001% and 10% w/w of the composition.

12. The composition of claim 11, wherein concentration of the personalizing substance in the composition is selected from the group consisting of .001%, 0.005%, 0.01%, 0.05%, 0.1%, 0.5%, 1%, 2%, 3%, 4%, 5%, 6%, 7%, 8%, 9% and 10% w/w of the composition.

13. The composition of claim 1, wherein the personalizing substance is in the form of microparticles and/or nanoparticles.

14. A method of delivering a personalizing substance to the skin of an individual comprising injecting into the individual's skin a composition comprising a personalizing substance wherein the personalizing substance is not encapsulated in a polymeric nano- or microparticle, and a carrier suitable for injection into the skin.

15. The method of claim 14, wherein injecting step is at the site of an existing tattoo on the individual's skin.

16. The method of claim 14, wherein the carrier is a tattoo ink.

17. The method of claim 16, further comprising the step of forming the composition by mixing the personalizing substance with the tattoo ink prior to injection.

18. The method of claim 16, wherein the step of injecting is repeated multiple times at different sites on the skin to form a tattoo design.

19. The method of claim 14, wherein the personalizing substance is in the form of nanoparticles and/or microparticles.

20. The method of claim 14, wherein the personalizing substance is selected from the group consisting of isolated DNA, sand, soil, metal, ceramics, and plant tissue.

21. The method of claim 19, wherein the personalizing substance is isolated DNA and wherein the DNA further comprises a personal identification characteristic selected from the group consisting of short tandem repeats (STRs), single nucleotide polymorphisms (SNPs), epigenetic markers, and methylated DNA patterns.
